(19) United States
(12) Patent Application Publication
(10) Pub. No.: US 2003/0065317 A1
(43) Pub. Date: Apr. 3, 2003

(54) Microwave ablation device

(76) Inventors: Eric N. Rudie, Maple Grove, MN (US); Stanley E. Kluge, Watertown, MN (US)

Correspondence Address: KINNEY & LANGE, P.A., THE KINNEY & LANGE BUILDING, 312 SOUTH THIRD STREET, MINNEAPOLIS, MN, 55415-1002, US

(21) Appl. No.: 10/247,080

(22) Filed: Sep. 19, 2002

Related U.S. Application Data
(60) Provisional application No. 60/323,491, filed on Sep. 19, 2001.
(60) Provisional application No. 60/338,250, filed on Nov. 2, 2001.

(51) Int. Cl. A61B 18/04

(52) U.S. Cl. 606/33

(57) ABSTRACT

A tissue ablation device includes a catheter shaft having an antenna lumen, an impedance-matched microwave antenna carried in the antenna lumen of the catheter shaft, at least one cooling lumen in the catheter shaft around the antenna lumen for circulation of cooling fluid, and a microwave generator operatively coupled to the antenna for energizing the antenna to create a lesion in the targeted tissue around the catheter shaft having a controlled location and size. In an exemplary embodiment, a tip is attached to an end of the catheter shaft for penetrating the tissue targeted for treatment. The device is effective for laparascopic or percutaneous procedures to treat tissues such as the kidney.

### CROSS-REFERENCE TO RELATED APPLICATION(S)

This application claims priority from U.S. Provisional Application No. 60/323,491 filed Sep. 19, 2001 for “Microwave Ablation Device” by E. Rudie and S. Kluge, and also claims priority from U.S. Provisional Application No. 60/338,250 filed Nov. 2, 2001 for “Microwave Ablation Device” by E. Rudie and S. Kluge.

### INCORPORATION BY REFERENCE

The aforementioned U.S. Provisional Application Nos. 60/323,491 and 60/338,250 are hereby incorporated by reference in their entirety.

### BACKGROUND OF THE INVENTION

The present invention relates to the field of microwave thermal ablation of tissue.

Surgical tissue ablation is becoming a popular tool for the treatment of benign and malignant tumors, through laparoscopic and percutaneous techniques, among others. Many ablative technologies have been employed in such treatments, including microwave thermotherapy, which operates to heat tissue above about 45° C. for a period of time sufficient to cause cell death and necrosis in a tissue region of interest. The therapeutic results of microwave ablation have been generally quite positive. However, in order for microwave ablation to become a truly effective tool for the laparoscopic and percutaneous treatment of tumors, an effective microwave antenna must be implemented to efficiently transfer energy to the targeted tissue region so that a precise lesion may be created of proper size and shape to destroy the tumor. In addition, a configuration that improves the achievable depth of heating would be desirable. There is a need in the art for a microwave ablation device having an efficient microwave antenna and a configuration that enables precise and effective ablation of a relatively large targeted region of tissue for the treatment of tumors.

### SUMMARY OF THE INVENTION

The present invention is a tissue ablation device that includes a catheter shaft having an antenna lumen, an impedance-matched microwave antenna carried in the antenna lumen of the catheter shaft, at least one cooling lumen in the catheter shaft around the antenna lumen for circulation of cooling fluid, and a microwave generator operatively coupled to the antenna for energizing the antenna to create a lesion in the targeted tissue around the catheter shaft having a controlled location and size. In an exemplary embodiment, a tip is attached to an end of the catheter shaft for penetrating the tissue targeted for treatment. The device is effective for laparascopic or percutaneous procedures to treat tissues such as the kidney.

### BRIEF DESCRIPTION OF THE DRAWINGS

FIG. 1A is a diagram illustrating the basic configuration for operation of a microwave ablation device according to the present invention.

FIG. 1B is a side view of an exemplary embodiment of the microwave ablation device of the present invention.

FIG. 2A is a partial section view of a microwave antenna according to the present invention.

FIG. 2B is an exploded view of a portion of the microwave antenna shown in FIG. 2A.

FIG. 2C is a partial section view of a microwave antenna employing a modified capacitor design according to the present invention.

FIG. 3A is a sectional view, and FIG. 3B is a perspective view with a cut-open region shown in section, of an uncooled version of a microwave ablation device according to a first embodiment of the present invention.

FIG. 4 is a diagram illustrating a heating pattern obtained during operation of an uncooled microwave ablation device in a tissue phantom.

FIG. 5A is a sectional view, and FIG. 5B is a perspective view with a cut-open region shown in section, of a cooled version of a microwave ablation device according to a second embodiment of the present invention.

FIG. 6 is a diagram illustrating a heating pattern obtained during operation of a cooled microwave ablation device in a tissue phantom.

FIG. 7A is a perspective view, and FIG. 7B is a side view, of an exemplary tip configuration for the microwave ablation device of the present invention.

FIG. 8 is a section view of an exemplary handle configuration for the microwave ablation device of the present invention.

FIG. 9 is a graph illustrating exemplary thermal history data obtained experimentally from ex vivo operation of a non-cooled microwave probe similar to that shown in FIGS. 3A and 3B.

FIG. 10 is a graph illustrating exemplary thermal history data obtained experimentally from ex vivo operation of a cooled microwave probe similar to that shown in FIGS. 5A and 5B.

### DETAILED DESCRIPTION OF THE PREFERRED EMBODIMENTS

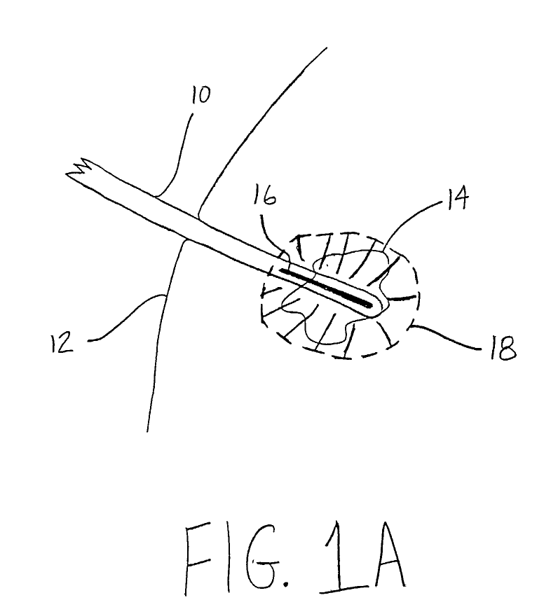

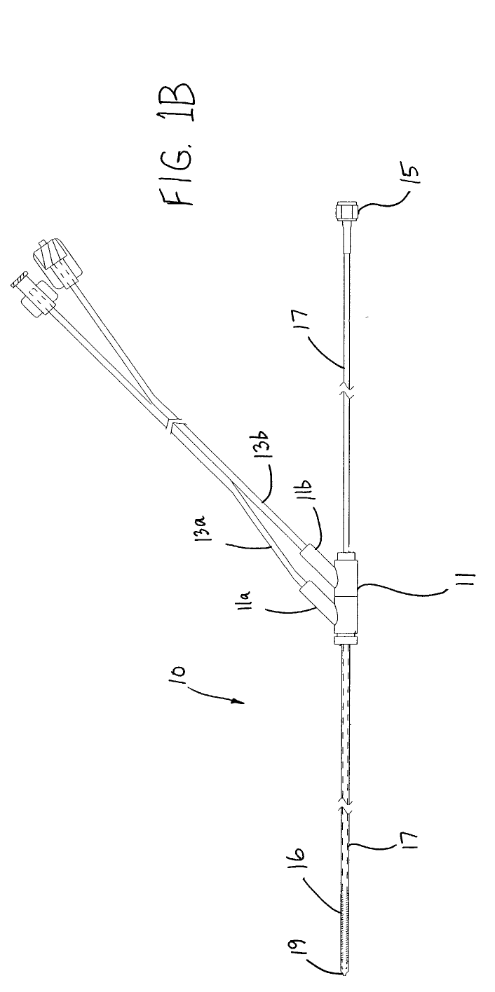

FIG. 1A is a diagram illustrating the basic configuration for operation of microwave ablation device 10 according to the present invention. In one embodiment, microwave ablation device 10 is inserted percutaneously through skin surface 12 into internal tissue that includes targeted tissue region 14, which may be a tumor or other tissue targeted for necrosis. In other embodiments, microwave ablation device may be inserted laparoscopically through a port, or may be used in an open surgical procedure. Microwave ablation device 10 includes microwave antenna 16, which is energized when positioned in targeted tissue region 14 to create lesion 18, which is a region of necrosis that encompasses the entirety of targeted tissue region 14.

FIG. 1B is a side view of an exemplary embodiment of microwave ablation device 10 of the present invention. Microwave ablation device 10 includes handle 11 having cooling fluid input/output ports 11a and 11b for communicating cooling fluid with tubes 13a and 13b. The device is connectable to a microwave power source through coupling 15. Microwave antenna 16 is carried at a distal end of microwave ablation device, connected to coaxial cable 17 which receives power from the microwave power source.

The impedance-matched microwave antenna employed by the present invention is configured as generally described in U.S. Pat. No. 5,300,099 entitled “Gamma Matched, Helical Dipole Microwave Antenna” and assigned to Urologix, Inc. U.S. Pat. No. 5,300,099, which discloses the impedance-matched microwave antenna in the context of a urethral catheter, and which is hereby incorporated by reference in its entirety. A brief description of the antenna is also included in this application for clarity and completeness.

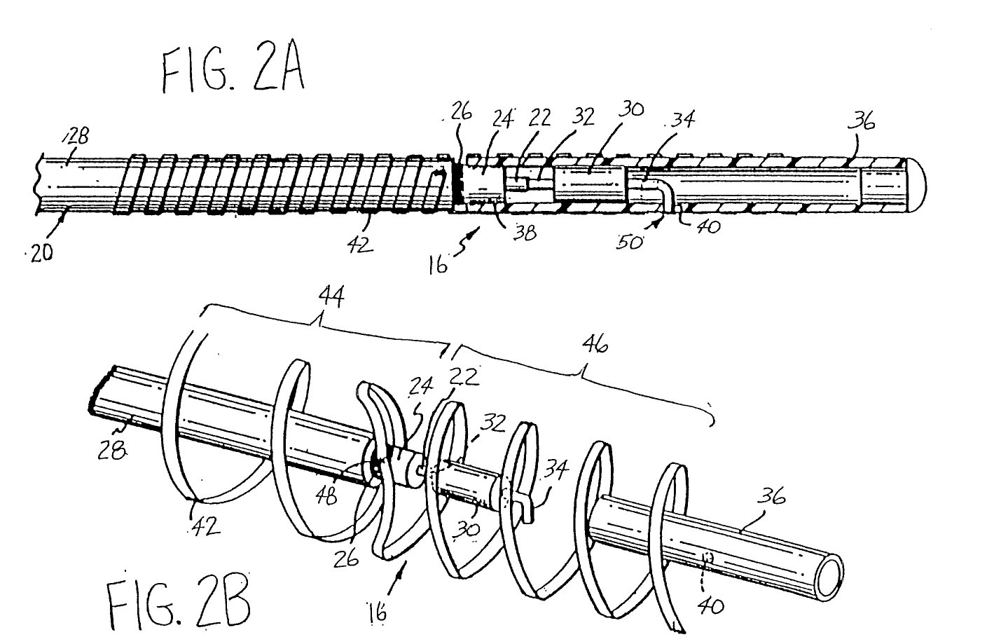

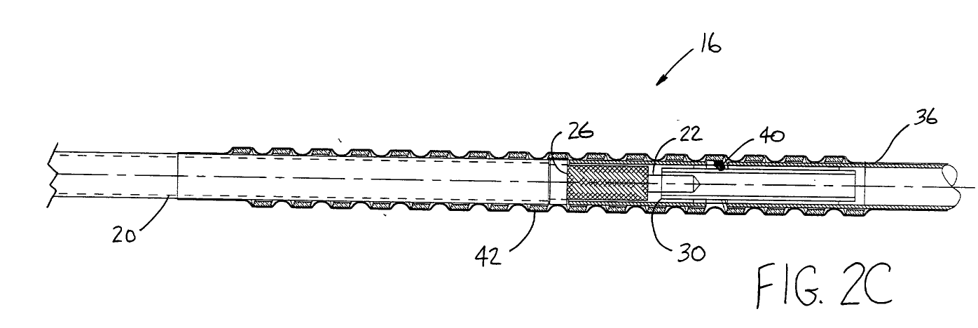

FIG. 2A is a partial sectional view of microwave antenna 16 according to the present invention. Antenna 16 is positioned at a distal-most end of shielded coaxial cable 20. In one exemplary embodiment, cable 20 is a standard RG 178U coaxial cable. In another embodiment, a semi-rigid coaxial cable with a solid outer conductor may be employed to provide additional stiffness. Cable 20 is preferably a non-paramagnetic, MRI-compatible cable, and includes inner conductor 22, inner insulator 24, outer conductor 26, and outer insulator 28. Outer insulator 28, outer conductor 26 and inner insulator 24 are stripped away to expose about 3 millimeters of outer conductor 26, about 1 millimeter of inner insulator 24 and about 1 millimeter of inner conductor 22. Capacitor 30 includes first end 32, which is connected to inner conductor 22 (such as by soldering, crimping or welding, for example), and second end 34, which is connected to antenna 16. Capacitor 30 serves to counteract a reactive component of antenna 16, thereby providing a 50 ohm impedance match between antenna 16 and coaxial cable 20 with the microwave generating source connected thereto.

Although capacitor 30 is shown in FIG. 2A as an axial-type metallized film component, it should be understood that a number of possible capacitor configurations may be used for the impedance matching of antenna 16. For example, a tubular ceramic capacitor or a discrete section of coaxial cable exhibiting the desired capacitance may be employed, as will be shown in the exemplary embodiment illustrated in FIG. 2C. Other possible capacitor configurations will be apparent to those skilled in the art.

Tubular extension 36, which is a hollow section of outer insulator 28 of coaxial cable 20, or a separate insulative piece approximating the dimensions of outer insulator 28, is positioned over capacitor 30 and the exposed length of inner insulator 24 and secured by bond 38. Tubular extension 36 includes hole 40, which provides an exit for second end 34 of capacitor 30 Wound about outer insulator 28 and tubular extension 36 is flat wire 42 Flat wire 42 is a single piece of flat copper wire with dimensions of about 0.009 inch by about 0.032 inch in cross-section, which provides a relatively large surface area for maximum current flow while minimizing the cross-sectional size of antenna 16.

FIG. 2B is an exploded view of a portion of antenna 16 which shows its helical dipole construction. Generally, the efficiency of any dipole antenna is greatest when the effective electrical length of the antenna is generally one half the wavelength of the radiation emitted in the surrounding medium. Accordingly, a relatively efficient simple dipole antenna, operating at about 915 MHz, would require a physical length of about 8 centimeters which, according to the present invention, would needlessly irradiate and damage healthy tissue outside of the targeted tissue. Furthermore, the physical length of a relatively efficient simple dipole antenna operating at about 915 MHz cannot be varied.

As shown in FIG. 2B, flat wire 42 is attached to outer conductor 26 at connection point 48. Flat wire 42 is then wound in a distal direction about outer insulator 28 and in a proximal direction about tubular extension 36, thereby forming first wire section 44 and second wire section 46, both of which are of equal length. In one embodiment, first and second wire sections 44 and 46 are each comprised of eight, equally-spaced windings of flat wire 42 The combined length of first and second wire sections 44 and 46, and hence the overall length of antenna 16, ranges from about 1 centimeter to about 6 centimeters, and varies according to the length of the area of targeted tissue which requires treatment. In an exemplary embodiment, silicone is applied around coaxial cable 20, capacitor 30 and flat wire 42, and a heat-shrink or chemical-shrink tubing is placed around the outside of antenna 16. After the tubing is shrunk to form a smooth outer surface, the silicone is exposed to ultraviolet radiation in order to cure the silicone and secure all of the components of antenna 16 in place. Other methods of securing antenna 16 in place and providing a smooth outer surface will be apparent to those skilled in the art.

The helical dipole construction of the present invention allows antenna 16 to range in physical length from about 1 to 6 centimeters, while electrically behaving like an eight centimeter-long simple dipole antenna. In other words, antenna 16 has an effective electrical length generally equal to one half of the wavelength of the radiation emitted in the surrounding medium, independent of its physical length. For purposes of definition, the surrounding medium includes the catheter shaft and the surrounding tissue. This is accomplished by varying the number and pitch of the windings of first and second wire sections 44 and 46 A family of catheters, which contain relatively efficient helical dipole antennas of different physical lengths, permits selection of the antenna best suited for the particular treatment area. In addition, antenna 16 of the present invention is capable of producing a constant heating pattern in tissue, concentrated about antenna 16, independent of the depth of insertion into the tissue.

Second end 34 of capacitor 30, which exits hole 40, is attached to second wire section 46 at tap point 50, as shown in FIG. 2A. Tap point 50 is a point at which the resistive component of the combined impedance of first wire section 44 and second wire section 46 matches the characteristic impedance of coaxial cable 20. The impedance of either first wire section 44 or second wire section 46 is expressed as Z, where Z=R+jX. The impedance Z varies from a low value at connection point 48 (FIG. 2B) to a high value at a point farthest from connection point 48. There exists a tap position where R is equal to 50 ohms, but an imaginary component, X, is inductive. This inductive component can be canceled by inserting a series capacitance, such as capacitor 30, which has a value of −jX ohms. This results in an impedance match of 50 ohms real. The resulting method of feeding antenna 16 is commonly called gamma matching. In one embodiment of the present invention, where the physical length of flat wire 42 is about 2.8 cm, tap point 50 is about 3.5 turns from connection point 48 on second wire section 46. In an exemplary embodiment, the value of capacitor 30 is about 2.7 pF.

FIG. 2C is a partial section view of microwave antenna 16 employing a modified capacitor design according to the present invention. Capacitor 30 is realized in this embodiment as a discrete section of coaxial cable exhibiting capacitance that is equal to the desired value for proper impedance matching, as described generally above. In the pictured embodiment, the coaxial cable section forming capacitor 30 is crimped onto inner conductor 22 of coaxial cable 20 and soldered to ensure a strong electrical and mechanical connection.

The helical dipole construction of antenna 16 achieves a relatively small size, which permits interstitial application. The helical dipole construction is also responsible for three features which enable antenna 16 to achieve greater efficiency than prior known interstitial microwave antennas: good impedance matching, good current carrying capability and an effective electrical length which is generally one half of the wavelength of the radiation emitted in the surrounding medium, independent of the physical length of antenna 16.

First, the good impedance match between antenna 16 and inner conductor 22 minimizes reflective losses of antenna 16, with measured reflective losses of less than 1% in an exemplary embodiment. Second, the use of flat ribbon wire 42 for first wire section 44 and second wire section 46 minimizes resistive losses of antenna 16 by providing a greater surface area upon which current can be carried. Finally, the helical dipole design of antenna 16 has an effective electrical length which is generally one half of the wavelength of the radiation emitted in the surrounding medium, independent of the physical length of antenna 16. This permits the physical length of antenna 16 to be varied to accommodate varying sizes of lesions while maintaining the same efficient, effective electrical length of antenna 16.

The use of an efficient microwave antenna is critical to the ability to focus thermal energy a distance from the antenna within a target volume. An inefficient antenna produces a lesser intensity of microwave radiation within the target volume than desired. The efficient helical dipole design of antenna 16 of the present invention ensures that almost all heat delivered during the treatment is delivered in the form of microwave energy, rather than conductive heat energy.

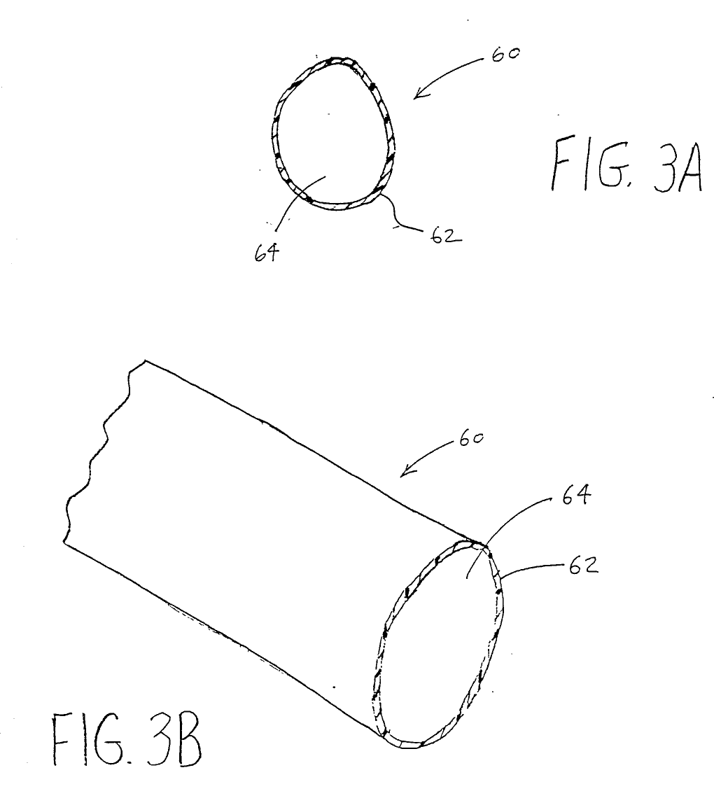

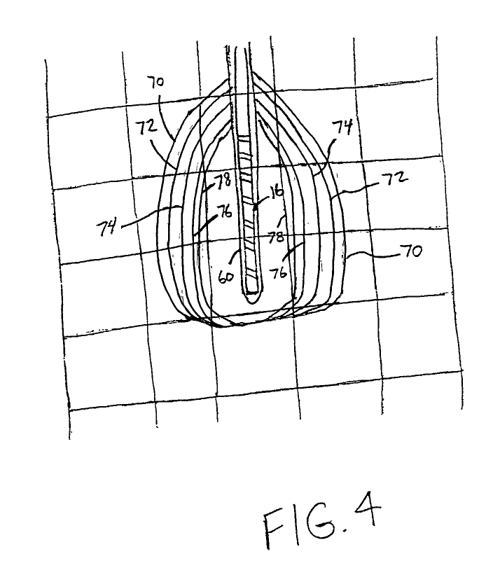

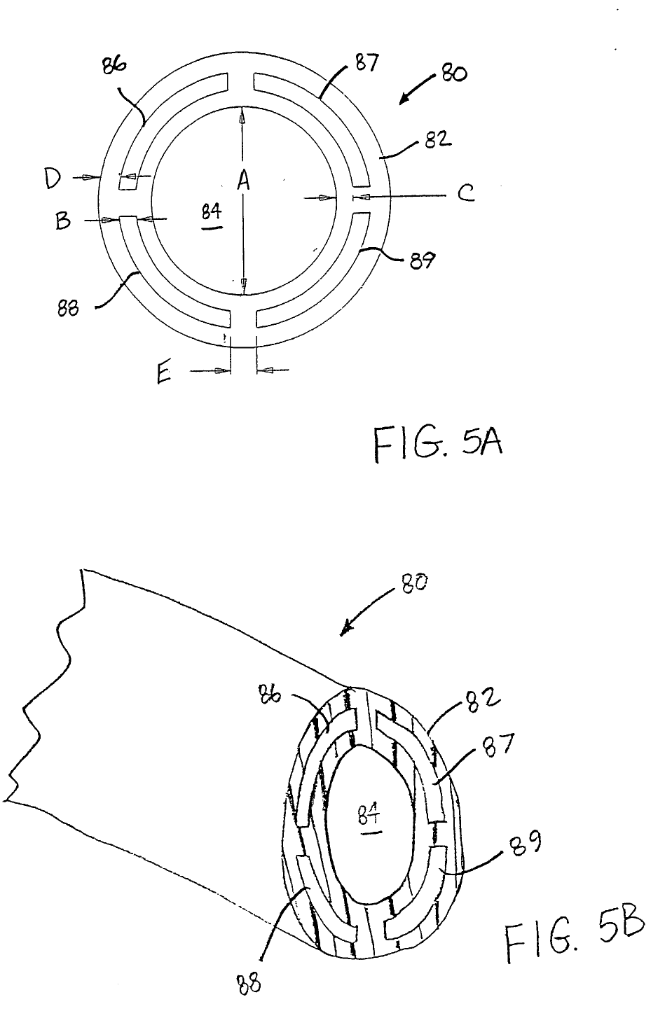

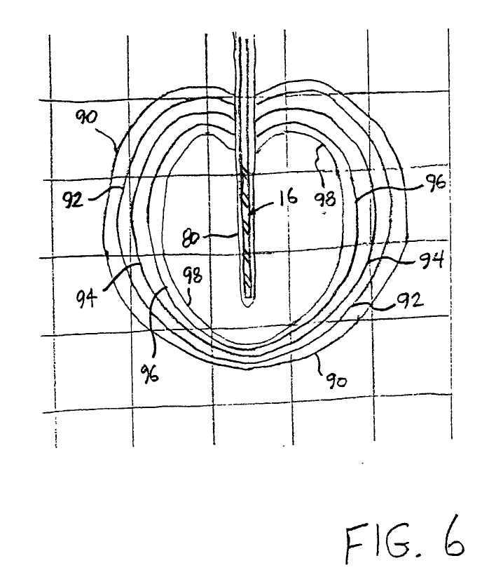

In order to create specific lesions sizes and shapes, a microwave ablation device may include only an energy-emitting microwave antenna, or may also include appropriately arranged cooling lumens for circulation of cooling fluid between the microwave antenna and the tissue being heated. A first embodiment of the present invention, described below with respect to FIGS. 3A, 3B and 4, is an uncooled microwave ablation device, while a second embodiment of the present invention, described below with respect to FIGS. 5A, 5B and 6, is a cooled microwave ablation device.

FIG. 3A is a sectional view, and FIG. 3B is a perspective view with a cut-open region shown in section, of catheter shaft 60 for realizing an uncooled version of a microwave ablation device according to a first embodiment of the present invention. Catheter shaft 60 is generally circular in cross-section, and includes outer wall 62 defining internal antenna lumen 64. Microwave antenna 16 (FIGS. 2A and 2B) is located in antenna lumen 64. In an exemplary embodiment, catheter shaft 60 includes a tip (not shown) that enables percutaneous or laparoscopic insertion of catheter shaft 60 into internal tissue, as is known in the art. Catheter shaft 60 has a length of about 30 centimeters (cm) and a diameter of less than 3 millimeters (mm) in an exemplary embodiment. Catheter shaft 60 preferably is sufficiently stiff to perforate soft tissue without buckling. Alternatively, catheter shaft 60 could be composed of a more flexible material if an appropriate introducer is provided to assist the insertion of catheter shaft 60 into tissue, or if a semi-rigid coaxial cable is used for the antenna or a stiffening element is employed to provide additional stiffness.

Microwave antenna 16 (FIGS. 2A and 2B) utilizes resonance to achieve an efficient and controlled transfer of energy from a transmission line such as a coaxial cable to the targeted tissue. The resonant frequency of microwave antenna 16 depends on the dielectric properties of the material surrounding it, with the highest dependence on the material closest to the antenna. Highly perfused tissue, such as a prostate or a kidney, for example, has a high water content, and water has a high dielectric constant. Therefore, the dielectric properties of these types of tissues are strongly influenced by the water content in the tissue. If water is driven out of the tissue by excessive heating, the dielectric properties of the tissue will change dramatically, causing the resonance of microwave antenna 16 to change to a point where microwave antenna 16 is incapable of continuing to achieve efficient transfer of energy. Therefore, in order to achieve deeper heating of tissue, it is necessary to maintain the temperature of tissue closest to the catheter shaft sufficiently low to maintain its water content and therefore its dielectric properties. In operating an uncooled microwave ablation device, temperatures are highest in the region closest to microwave antenna 16, and drop off with increasing distance from microwave antenna 16. The above-described need to keep temperatures adjacent to the catheter below about 100° C. results in a limited depth in which tissue heating capable of cell death (typically greater than about 45-50° C., depending on treatment time) can occur.

FIG. 4 is a diagram illustrating a heating pattern obtained during operation of an uncooled microwave ablation device in a tissue phantom, utilizing catheter shaft 60 configured as shown in FIGS. 3A and 3B. The grid lines in FIG. 4 are spaced 1 cm apart. Upon energization of microwave antenna 16 with an input power of 10 Watts for an exposure time of 10 minutes, a heating pattern was observed as shown in FIG. 4. Specifically, 30° C. isotherm 70, 35° C. isotherm 72, 40° C. isotherm 74, 45° C. isotherm 76 and 50° C. isotherm 78 represent the temperature rise above baseline in the heating pattern achieved. During the operation shown in FIG. 4, water on the surface of catheter shaft 60 was just beginning to boil, indicating that the heating pattern achieved is nearly the maximum heating possible without adversely affecting the dielectric constant of the tissue phantom and therefore inhibiting the resonant performance of microwave antenna 16. The diagram of FIG. 4 shows that the uncooled microwave ablation device is able to achieve temperatures above about 45° C. at a radial distance of about 0.6 cm from the outer surface of catheter shaft 60 on each side, producing a total lesion diameter of about 1.5 cm (since catheter shaft 60 has a diameter of about 0.3 cm). It will be understood by those skilled in the art that other geometrical configurations and variation of the treatment parameters may result in the creation of lesions of larger or smaller sizes.

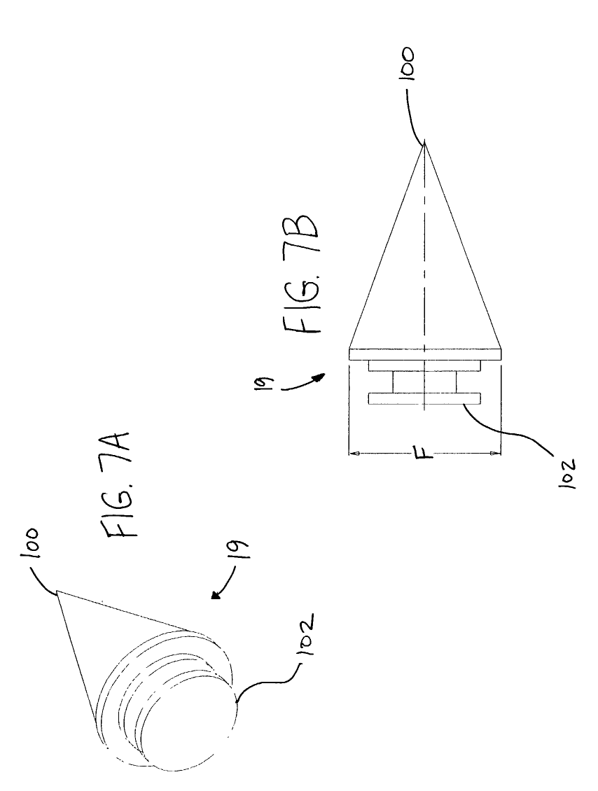

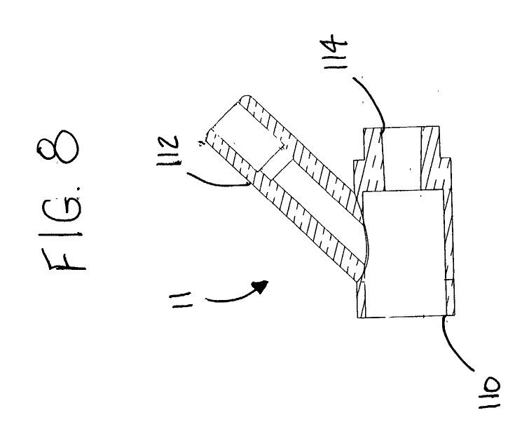

FIG. 5A is a sectional view, and FIG. 5B is a perspective view with a cut-open region shown in section, of catheter shaft 80 for realizing a cooled version of a microwave ablation device according to a second embodiment of the present invention. Catheter shaft 80 is generally circular in cross-section, and includes walls 82 defining internal antenna lumen 84 and cooling lumens 86, 87, 88 and 89. In a first exemplary embodiment, the outer diameter of catheter shaft is about 4.75 millimeters (mm), the diameter of antenna lumen 84 (dimension A) is about 2.54 mm, the thicknesses of cooling lumens 86, 87, 88 and 89 (dimension B) are about 0.76 mm, and the wall thickness between antenna lumen 84 and cooling lumens 86, 87, 88 and 89 (dimension C), between cooling lumens 86, 87, 88 and 89 and catheter shaft 80 (dimension D), and between each of cooling lumens 86, 87, 88 and 89 (dimension E) are about 0.12 mm. In a second exemplary embodiment, a smaller catheter is employed, and the outer diameter of catheter shaft is about 3.45 millimeters (mm), the diameter of antenna lumen 84 (dimension A) is about 2.54 mm, the thicknesses of cooling lumens 86, 87, 88 and 89 (dimension B) are about 0.20 mm, and the wall thickness between antenna lumen 84 and cooling lumens 86, 87, 88 and 89 (dimension C), between cooling lumens 86, 87, 88 and 89 and catheter shaft 80 (dimension D), and between each of cooling lumens 86, 87, 88 and 89 (dimension E) are about 0.12 mm. Microwave antenna 16 (FIGS. 2A and 2B) is located in antenna lumen 84. Cooling fluid, such as ionized water in one embodiment, is circulated through cooling lumens 86, 87, 88 and 89 in a manner generally known in the art. An example of a suitable cooling system is disclosed in the context of a urethral catheter in U.S. Pat. No. 5,300,099 entitled “Gamma Matched, Helical Dipole Microwave Antenna” and assigned to Urologix, Inc., which has been incorporated by reference herein. In one exemplary embodiment, cooling fluid is circulated into cooling lumens 86 and 87 and exits from cooling lumens 88 and 89. In such an embodiment, cooling lumens 86 and 87 communicate with cooling lumens 88 and 89 near the distal end of catheter shaft 80 to provide a continuous fluid communication path in catheter shaft 80. Alternatively, cooling lumens 86, 87, 88 and 89 may be configured with any other combination of fluid flow patterns, as is known in the art. In an exemplary embodiment, catheter shaft 80 includes a tip (shown in detail in FIGS. 7A and 7B) that enables percutaneous or laparoscopic insertion of catheter shaft 80 into internal tissue, as is generally known in the art. Catheter shaft 80 has a length of about 30 centimeters (cm) in an exemplary embodiment. Catheter shaft 80 preferably is sufficiently stiff to perforate soft tissue without buckling. Alternatively, catheter shaft 80 could be composed of a more flexible material if an appropriate introducer is provided to assist the insertion of catheter shaft 80 into tissue, or if a semi-rigid coaxial cable is used for the antenna or a stiffening element is employed to provide additional stiffness.

FIG. 6 is a diagram illustrating a heating pattern obtained during operation of a cooled microwave ablation device in a tissue phantom, utilizing catheter shaft 80 configured as shown in FIGS. 5A and 5B. The grid lines in FIG. 6 are spaced 1 cm apart. Upon energization of microwave antenna 16 with an input power of 45 Watts for an exposure time of 10 minutes, with coolant at 20° C. circulated through cooling lumens 86, 87, 88 and 89 (FIGS. 5A and 5B), a heating pattern was observed as shown in FIG. 6. Specifically, 30° C. isotherm 90, 35° C. isotherm 92,40° C. isotherm 94, 45° C. isotherm 96 and 50° C. isotherm 98 represent the temperature rise above baseline in the heating pattern achieved. During the operation shown in FIG. 6, there was no evidence of boiling water on the surface of catheter shaft 80, indicating that the temperature of tissue adjacent to catheter shaft 80 was maintained below a boiling threshold and the resonant operation of microwave antenna 16 was not adversely affected by any change in the dielectric properties of the tissue surrounding catheter shaft 80. This suggests that even greater depths of high temperature fields may be created by the application of higher power to microwave antenna 16. The diagram of FIG. 6 shows that the cooled microwave ablation device is able to achieve temperatures above about 45° C. at a radial distance of about 1.2 cm from the outer surface of catheter shaft 60, producing a total lesion diameter of about 2.7 cm (since catheter shaft 80 has a diameter of about 0.5 cm, although the drawing in FIG. 6 is not necessarily shown to scale). The cooled version of the microwave ablation device may achieve lesions having diameters exceeding about 4 cm in some embodiments.

FIG. 7A is a perspective view, and FIG. 7B is a side view, of tip 19 for use with the microwave ablation device of the present invention. Tip 19 includes a pointed piercing portion 100 and a mounting portion 102. Tip 19 has a diameter (dimension F) that matches the outer diameter of the catheter shaft. Mounting portion 102 of tip 19 is configured to allow the cooling lumens of the catheter shaft to communicate with one another so that cooling fluid is able to circulate along the length of catheter shaft in the cooling lumens in both a feed path and a return path. In the exemplary embodiment illustrates in FIGS. 7A and 7B, piercing portion 100 of tip 19 is configured with sufficient stiffness, strength and sharpness to pierce into a targeted tissue region such as a kidney. The suitable materials for providing this capability are generally known in the art. In other embodiments, tip 19 may be blunt, with insertion achieved by other complementary surgical tools generally known and available to those skilled in the art. In either case, the microwave ablation device of the present invention is a “surgical” device in that it is directly inserted into targeted tissue without using a natural body lumen or cavity.

FIG. 8 is a section view of handle 11 for use with the microwave ablation device of the present invention. Handle 11 includes a catheter retaining portion 110 and a cooling fluid input/output portion 112. A coaxial cable (not shown) is inserted into handle 11 at cable input aperture 114, and is received into the catheter shaft inside catheter retaining portion 110. Cooling fluid flows through a tube (not shown) which is received by cooling fluid input/output portion 112 of handle II, and enters the catheter shaft inside catheter retaining portion 110. Handle 11 thus provides an effective manifold system for receiving the components of the interior portions of the catheter shaft. In an exemplary embodiment, handle 11 can be formed by injection molding, or may be a two-piece “clamshell” construction similar to the handle disclosed in U.S. application Ser. No. 09/733,109 filed Dec. 8, 2000 for “Thermal Therapy Catheter” by E. Rudie, S. Stockmoe, A. Hjelle, B. Ebner, J. Crabb, J. Flachman, S. Kluge, S. Ramadhyani and B. Neilson, which is hereby incorporated by reference.

The embodiment illustrated in FIG. 8 shows cooling fluid input/output portion 112 of handle 11 departing at an acute angle of about 45 degrees. Other embodiments of handle 11 may employ different acute angles or an obtuse angle of departure, to vary the forces experienced during operation of the microwave ablation device for maximum ease of use by a physician.

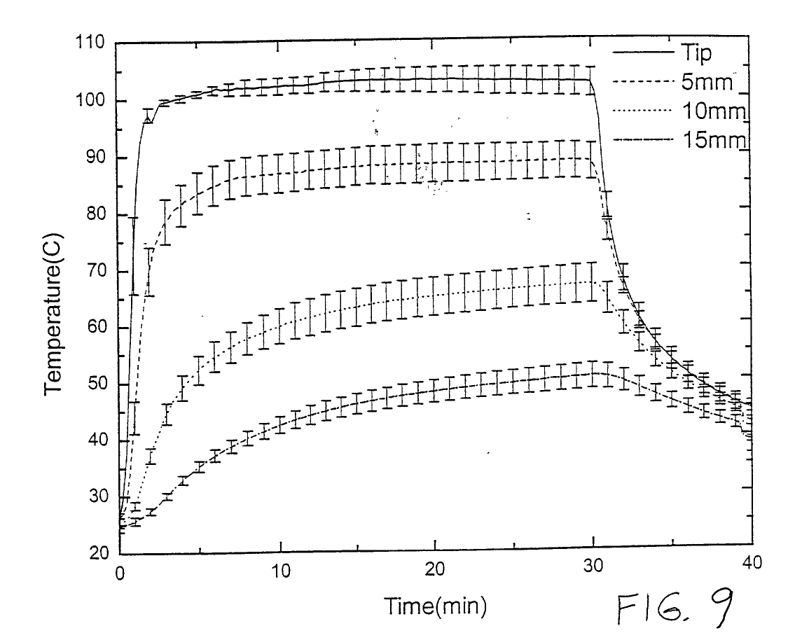

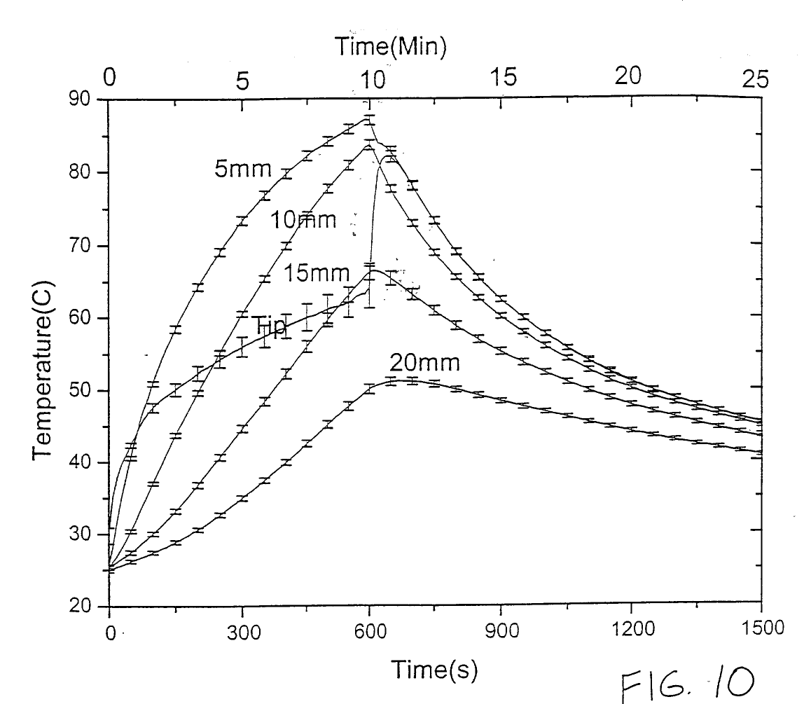

FIG. 9 is a graph illustrating exemplary thermal history data obtained experimentally from ex vivo operation of a non-cooled microwave probe similar to that shown in FIGS. 3A and 3B. The probe was operated for 30 minutes at a power level of 10-20 Watts such that the temperature at the tip of the probe remained constant. The temperatures at the probe tip and at radial distances 5 millimeters (mm), 10 mm and 15 mm from the tip were measured. The error bars on the graph represent the Standard Error of the Mean (SEM) of the measurements.

FIG. 10 is a graph illustrating exemplary thermal history data obtained experimentally from ex vivo operation of a cooled microwave probe similar to that shown in FIGS. 5A and 5B. The probe was operated for 10 minutes at a constant power level of 50 Watts with a coolant temperature of 37° C. (both power and cooling were discontinued after 10 minutes). The temperatures at the probe tip and at radial distances 5 millimeters (mm), 10 mm and 15 mm from the tip were measured. The error bars on the graph represent the Standard Error of the Mean (SEM) of the measurements.

A number of observations can be made about the measured thermal history data of FIGS. 9 and 10. The depth of high temperature heating achieved by the uncooled probe (as shown in FIG. 9) is less than the depth of high temperature heating achieved by the cooled probe (as shown in FIG. 10). This is primarily because of the reduction in power that is required to keep the temperature at the catheter shaft below about 95° C. to avoid tissue charring. Also, it should be realized that the peak temperature achieved by the uncooled probe during true in vivo operation will be somewhat lower than the peak temperature achieved by the uncooled probe during ex vivo operation (as shown in FIG. 10), due to the cooling effect of blood perfusion that occurs in vivo. However, despite the lower peak temperature, testing has shown that effective high temperature heating can be achieved at significant, controlled depth during the in vivo procedure, validating the efficacy of the present invention. An example of in vivo testing results is described below.

### In Vivo Testing

Clinical trials were performed to evaluate the performance of the microwave ablation device of the present invention. Implantation of the device was made 26 mm into the lateral cortex of in vivo perfused porcine kidneys. A 3.5 mm non-cooled probe generally similar to that shown in FIGS. 3A and 3B was operated for eight samples, with power of 10-15 Watts maximum, adjusted to maintain the probe tip temperature below 95° C. The non-cooled probe was operated for 30 minutes. A 4.75 mm water cooled probe generally similar to that shown in FIGS. 5A and 5B was operated for five samples, with power of constant 50 Watts at 37° C. coolant temperature. The cooled probe was operated for 10 minutes. The kidneys were resected 3 hours after treatment and bisected for evaluation with gross measurements made 1.0 cm below the capsular surface.

Well-delineated lesions were produced with an inner zone of complete ablation and outer transition zone (see Table 1 below). Both probes were associated with minimal intraoperative hemorrhage (less than 20 cc) and maintained tissue integrity without parenchymal cracking. Neither probe showed renal artery nor vein thrombosis within the post-treatment perfusion period. While some tissue charring was identified with the non-cooled probe, it was not seen in the kidneys treated with the cooled probe. The cooled probe resulted in an enlarged ablation zone and reduced the treatment time needed without an apparent increase in procedural complications.

TABLE 1

| Probe Type | Treatment Time | Total Diameter | Inner Zone Diameter |
|---|---|---|---|
| Non-cooled | 30 minutes | 1.8 ± 0.3 cm | 1.2 + 0.2 cm |
| Cooled | 10 minutes | 3.4 ± 0.5 cm | 1.8 ± 0.3 cm |

The present invention is a microwave ablation device for controllably creating thermal lesions to treat tissue. The impedance-matched antenna employed by the device reduces reflective losses and provides optimal performance in controlling the size and shape of the thermal field generated by the device to treat a targeted region of tissue. While either cooled or non-cooled embodiments of the microwave ablation device may be used with beneficial effect, the cooled embodiment provides the ability to create a larger lesion due to its ability to avoid defecation of tissue in the vicinity of the probe that prevents deep heating. The cooling is not used to preserve tissue adjacent to the probe or to avoid patient pain (which are the traditional uses of cooling), but instead serves to increase the size of the tissue region that is thermally damaged, including the tissue directly adjacent to the probe. The size of the catheter shaft and the cooling lumens can also be varied, yielding variations in lesion sizes and in other therapy parameters.

Although the present invention has been described with reference to preferred embodiments, workers skilled in the art will recognize that changes may be made in form and detail without departing from the spirit and scope of the invention.

What is claimed is:

1. A surgical tissue ablation device comprising:
a catheter shaft having an antenna lumen;
an impedance-matched microwave antenna carried in the antenna lumen of the catheter shaft;
at least one cooling lumen in the catheter shaft around the antenna lumen for circulation of cooling fluid; and
a microwave generator operatively coupled to the antenna for energizing the antenna to create a lesion in tissue targeted for treatment around the catheter shaft having a controlled location and size.

2. The tissue ablation device of claim 1, further comprising:
a tip attached to an end of the catheter shaft for penetrating the tissue targeted for treatment; and

3. The tissue ablation device of claim 1, wherein the catheter shaft has an outer diameter of about 4.75 millimeters (mm), the antenna lumen has a diameter of about 2.54 mm, the at least one cooling lumen has a thickness of about 0.76 mm, and a wall thickness around the at least one cooling lumen is about 0.12 mm.

4. The tissue ablation device of claim 1, wherein the catheter shaft has an outer diameter of about 3.45 millimeters (mm), the antenna lumen has a diameter of about 2.54 mm, the at least one cooling lumen has a thickness of about 0.20 mm, and a wall thickness around the at least one cooling lumen is about 0.12 mm.

5. The tissue ablation device of claim 1, wherein the at least one cooling lumen comprises four cooling lumens around the antenna lumen.

6. A method of thermally treating tissue comprising:
penetrating tissue targeted for treatment with a catheter shaft carrying an impedance-matched microwave antenna; and
energizing the microwave antenna to create a lesion in the targeted tissue having a controlled location and size; and
circulating cooling fluid around the microwave antenna while energizing the microwave antenna to create the lesion.

7. The method of claim 6, wherein the step of penetrating targeted tissue comprises:
laparascopically inserting the catheter shaft through a port into the targeted tissue.

8. The method of claim 6, wherein the step of penetrating targeted tissue comprises:
percutaneously inserting the catheter shaft through skin into the targeted tissue.

9. The method of claim 6, wherein the step of energizing the microwave antenna is performed for no greater than about 10 minutes.

10. The method of claim 6, wherein the lesion has a total diameter greater than about 2 centimeters.

11. The method of claim 6, wherein the cooling fluid has a temperature of about 37° C.

12. The method of claim 6, wherein the step of energizing the microwave antenna comprises delivering a constant power of about 50 Watts.
